(19) United States
(12) Patent Application Publication
(10) Pub. No.: US 2013/0122492 A1
(43) Pub. Date: May 16, 2013

(54) DETECTION, ISOLATION AND ANALYSIS OF RARE CELLS IN BIOLOGICAL FLUIDS

(71) Applicants: Javad Khosravi, Toronto (CA); Leonard H. Kellner, Massapequa, NY (US); Hassan Benanni, Dix Hills, NY (US)

(72) Inventors: Javad Khosravi, Toronto (CA); Leonard H. Kellner, Massapequa, NY (US); Hassan Benanni, Dix Hills, NY (US)

(73) Assignee: KELLBENX INC., Great River, NY (US)

(21) Appl. No.: 13/295,532

(22) Filed: Nov. 14, 2011

(51) Int. Cl. C12Q 1/68; G01N 33/566; C12N 11/02

(52) U.S. Cl. 435/6.11; 435/177; 435/7.21; 435/6.1

(57) ABSTRACT

The invention provides a method for isolating or enriching a rare cell from a biological fluid of a mammal employing an antibody that binds a cell-surface antigen of the rare cell. The immobilized antibody is incubated with a sample of biological fluid that includes the rare cells and a plurality of other cells so as to form an antibody-rare cell complex. The complex can be detected or isolated and subsequently analyzed by any of a variety of physical, chemical and genetic techniques.

### FIELD OF THE INVENTION

The present invention relates to immunological methods and kits for detection, capture and isolation of rare cells from biological fluids for analysis of their antigenic, phenotypic and genetic characteristics. In particular, the invention provides methods and kits for detection, capture, isolation and analysis of fetal nucleated red blood cells (NRBCs) from maternal blood.

### BACKGROUND

The practice of prenatal diagnosis to detect possible chromosomal and genetic abnormalities of the fetus enables parents and caregivers to initiate monitoring of predispositions and early treatment of diseases or conditions. The practice of prenatal diagnosis has been established to detect possible chromosomal and genetic abnormalities of the fetus, thus enabling informed decisions by the parents and the care givers. Among various chromosomal abnormalities compatible with life (1) (aneuploidy 21, 18, 13, X, Y), Down syndrome (DS), caused by the presence of all or part of an extra copy chromosome 21, is the most common genetic cause of mental retardation and the primary reason for women seeking prenatal diagnosis (1, 2). Although definitive detection of chromosomal abnormalities and singe gene disorders is possible by karyotype analysis of fetal tissues obtained by chorionic villus sampling (3), amniocentesis (3, 4) or umbilical cord sampling (5), these procedures are highly invasive, require skilled professionals, and are prone to significant risk of fetal loss (up to 1%) and/or maternal complications (3-5). Cytogenetic disorders are reportedly occurs in about 1% of live births, 2% of pregnant women older than 35 years, and in approximately 50% of spontaneous first trimester miscarriage (6). The incidence of single gene defects in a population of one million live births is reportedly about 0.36% (7).

To minimize risks in conditions such as DS, these invasive, but definitive, tests are offered to women identified by a set of screening criteria as having the highest risk for fetal chromosomal abnormalities. This group generally includes pregnancies with maternal age of 35 years of age or older and abnormal responses to ultrasound examinations of the fetus and/or maternal serum marker screening tests performed during first and/or second trimesters of pregnancy (8). The preferred first trimester screening, involving quantification from serum of PAPP-A (pregnancy-associated plasma-protein-A), free β-hCG (free β-human chorionic gonadotrophins), and ultrasound examination of nuchal translucency, has DS detection rate of about 90%, but at the expense of significant 5% false positive rate (8). A recent met-analysis of first trimester screening studies (9) concluded that in practice the achievable sensitivity might be significantly lower (about 80-84%) than reported. The problems of poor performance, particularly in light of screen-positive rate of 5%, invariably results in high rates of unnecessary and costly invasive confirmatory testing and thus, increased risks to the developing pregnancies.

The apparent limitations have been the primary social, scientific, and economic motivations for seeking alternative strategies. The latter has been reinforced by the rise in occurrence of DS, due mainly to increasing trend in maternal age at pregnancy, without comparable increases in birth rate (10). The recent guideline by the American College of Obstetricians and Gynecologists (ACOG) advising its members to test all expected mothers for genetic abnormalities (11) is further indication of the unmet need for non-invasive technologies that could safely lead to specific diagnosis of fetal genetic status. Accordingly, development of non-invasive prenatal diagnostics has become one of the most aggressively contested fields in modern day medicine (12). The candidate strategies are expected to encompass all of the advantages of existing invasive methods so that they could function as a stand-alone non-invasive diagnostic test or be used as highly accurate confirmatory test for analysis of the high numbers of false positives associated with current screening practices (13). The new testing strategies should, in addition, address analytical, manufacturing, and operational complexities such that the new methods provide a reliable, simple, and cost effective alternative.

For several decades, the search for non-invasive alternatives has focused on isolation, identification, and subsequent analysis of fetal genetic materials that normally cross the placental barrier into maternal circulation. Since the pioneering reports on detection of fetal cells in 1893 (14) and later of fetal cell-free DNA (15) and RNA (16) in maternal blood, two promising approaches based on analysis of fetal cells or cell free fetal genetic materials has received tremendous interest. In comparison to cell-free fetal DNA or RNA, intact fetal cells can provide access to complete fetal genetic materials important for detection of chromosomal abnormalities as well as a more complete assessment of fetal genetic status (17). Because of relative increase in number of fetal cells in pregnancies complicated by chromosomal abnormality or in conditions such as preeclampsia (18), a reliable isolation method would likely lend itself to development of novel non-invasive diagnostic methods for these conditions based on fetal cell enumeration and/or quantification of the cell detection signal.

A number of significant challenges have hampered development of reliable fetal cell isolation methods. The reported rarity of occurrences at approximately one to two cells per milliliter (mL) of maternal blood has been considered a formidable barrier to reproducible isolation of fetal cells with sufficient purity and yield (18). A successful cell isolation strategy would therefore require exceptional efficiency, sensitivity, and specificity. It is possible that the number of fetal cells entering maternal circulation is significantly higher than previously believed, as reported numbers have been so far obtained by inefficient multi-step technologies that are prone to poor yield and cell loss. Among variety of candidate fetal cells (19) (trophoblasts, lymphocytes, nucleated red blood cells, and hematopoietic stem cells), nucleated red blood cells (NRBC), known also as erythroblasts, have most of the desired characteristics. Fetal NRBCs have limited life span and proliferative capacity, are mononucleated, carry a representative complement of fetal chromosomes, and are consistently present in maternal blood (17-20). Studies of fetal erythropoiesis have, however, identified two distinct processes, occurring initially in yolk sack (primitive erythropoiesis, producing primitive erythroblasts) and subsequently in fetal liver and bone marrow (producing definitive erythroblasts) (17). Both primitive and definitive erythroblasts have been detected in maternal circulation, but their exact time of appearance, their relative numbers, and distribution throughout pregnancy has not been clearly defined. However, while primitive erythroblasts are the predominant first trimester cell type, they are progressively replaced by the definitive type that persists until term (17, 20).

Primitive erythroblasts have distinguishing morphological features of having a high cytoplasmic to nuclear ratio, comparatively larger size, and containing an embryonic type of hemoglobin know as ε-globulin (17, 20). Collectively, the above characteristics and knowledge of differential expression of various cell surface markers such as cluster of differentiation (CD) markers (CD34, CD35, CD36, CD45, CD 47, CD71), glycophorin-A, and i-antigen (17, 20, 21), has identified primitive erythroblasts as an ideal first trimester target.

Epsilon-positive erythroblasts in fetal blood decline linearly from seven weeks, reaching negligible numbers by about 14 weeks of gestation (22). On the other hand, a recent report suggests definitive erythroblasts are enucleated before entering circulation (17) and if substantiated, then first trimester primitive erythroblasts would remain the only useful target. Epsilon globulin is reportedly a highly specific primitive fetal erythroblast identifier (20, 22).

Current approaches to non-invasive prenatal diagnosis has been based on exploiting physical, structural, morphological, and antigenic attributes of target cells and the process has so far engaged three independent steps (22, 23). These are: (1), development of technologies designed for enrichment of fetal cells from maternal blood (2), identification of fetal cells among the heterogeneous mixture of enriched cells and (3), genetic analysis of the identified cells by chromosomal fluorescence in situ hybridization (FISH), various PCR techniques and/or gene sequencing before and/or after micromanipulation of the targets (17, 21-23). In attempts to minimize current complexities, inefficiencies, and inconsistencies approaches that combine fetal cell identification step with molecular genetics-based diagnosis have been also considered (22).

Inadequacies of the current fetal cell isolation strategies have been identified by a recent review (18) as the major factor limiting development of a reliable non-invasive prenatal diagnostic method. Currently, the most commonly explored fetal cell enrichments include multi-step combinations of selective erythrocyte lysis, density gradient centrifugation, charge flow separation, fluorescent-activated cell sorting (FACS), and magnetic-activated cell sorting (MACS) (18, 23). Newer alternatives include more complex approaches based on microelectronic mechanical systems (MEMS) and/or automation of some of the current cell enrichment methods in combination with morphological differences, immunophenotyping and/or micromanipulation of the identified cells (18, 24-28).

It is now apparent that development of simple, sensitive, and specific fetal cell isolation technology capable of high efficiency and consistency is an absolute pre-requisite to developing successful non-invasive prenatal tests for practical use. The fact that the latter has not been as yet realized despite availability of downstream technologies (FISH, PCR, and genomic sequencing) for accurate detection of genetic and chromosomal abnormalities is a reflection of significant inadequacies of the currently available cell isolation methods (17, 18, 21, 23).

There remains an urgent need for a novel simple, fast and reliable fetal NRBC isolation technology to overcome this widely acknowledged formidable obstacle (17, 18, 20-28). Ideally, such a kit that addresses these needs for detection, isolation and analysis of fetal NRBCs should be cost effective to manufacture, while maintaining high isolation sensitivity, specificity, and consistency.

The present invention provides such a novel simple, fast, and reliable fetal NRBC isolation kit based on a technology that overcomes these obstacles. The kit can be manufactured cost effectively while maintaining high isolation sensitivity, specificity, and consistency.

### SUMMARY OF THE INVENTION

The present invention fulfills an unmet urgent need for a reliable technology and associated protocols to provide methods for detection, enrichment and isolation of rare cells from biological fluids. The invention further provides a system and associated methods that function as an integral part of a standalone kit for fetal NRBC isolation, identification and subsequent analysis of specific fetal genetic abnormalities or testing for presence of any of a panel of fetal genetic abnormalities and other genotypes of diagnostic interest. The invention also addresses unmet needs for reliable rare cell isolation methods in other fields that are currently faced with similar detection and analysis limitations, such as circulating stem cells and tumor cells.

The invention provides a method of enriching and/or isolating a rare cell from a biological fluid of a mammal; the method includes: (i) providing an antibody immobilized on a substrate, wherein the antibody binds a cell-surface antigen of the rare cell; (ii) contacting the immobilized antibody with a sample of biological fluid, wherein the bodily fluid contains the rare cell and a plurality of other cells; (iii) incubating the immobilized antibody with the sample of bodily fluid under conditions suitable for binding of the antibody to the cell-surface antigen of the rare cell so as to form an antibody-rare cell complex; and (iv) washing the antibody-rare cell complex to remove the unbound cells and provide an immobilized antibody-rare cell complex.

The invention also provides a method of detecting a rare cell in a biological fluid; the method includes: (i) providing a first antibody immobilized on a substrate, wherein the first antibody binds a first cell-surface antigen of the rare cell; (ii) contacting the immobilized first antibody with a sample of biological fluid, wherein the bodily fluid contains the rare cell and a plurality of other cells; (iii) incubating the immobilized first antibody with the sample of bodily fluid under conditions suitable for binding of the first antibody to the first cell-surface antigen of the rare cell so as to form a first antibody-rare cell complex; (iv) washing the first antibody-rare cell complex to remove the unbound cells and provide an isolated first antibody-rare cell complex; (v) incubating the first antibody-rare cell complex with a second antibody that binds a second cell-surface antigen of the rare cell under conditions suitable for binding of the second antibody to the a second cell-surface antigen in order to form a first antibody-rare cell-second antibody complex; and (vi) detecting the second antibody in the first antibody-rare cell-second antibody complex and thereby detecting the presence of the rare cell in the sample of the bodily fluid.

The invention further provides a method of detecting a rare cell in a biological fluid; the method includes: (i) providing a first antibody immobilized on a substrate, wherein the first antibody binds a first cell-surface antigen of the rare cell; (ii) contacting the immobilized first antibody with a sample of biological fluid, wherein the bodily fluid contains the rare cell and a plurality of other cells; (iii) incubating the immobilized first antibody with the sample of bodily fluid under conditions suitable for binding of the antibody to the cell-surface antigen of the rare cell so as to form a first antibody-rare cell complex and a plurality of unbound cells; (iv) washing the first antibody-rare cell complex to remove the unbound cells; (v) lysing the rare cells of the first antibody-rare cell complex to form a lysate that contains a rare cell-specific nucleic acid sequence and incubating the lysed cells with a nucleic acid probe that is complementary to the rare cell-specific nucleic acid sequence under conditions suitable for hybridization of the nucleic acid probe with the rare cell-specific nucleic acid sequence in order to form a double stranded complex; and (vi) detecting the double stranded complex and thereby detecting the presence of the rare cell in the sample of the bodily fluid.

The invention also provides a kit for detection or isolation of a rare cell from a biological fluid, such as for instance, a fetal cell from maternal blood; the kit includes an antibody immobilized on a substrate wherein the antibody is specific for a cell-surface antigen of the rare cell; and a buffer solution suitable for antigen antibody binding.

The invention provides a method of estimating the number of rare cells per unit of a biological fluid of a mammal; the method includes: (i) providing an antibody immobilized on a substrate, wherein the antibody binds a cell-surface antigen of the rare cell; (ii) contacting the immobilized antibody with a known unit sample of biological fluid, wherein the bodily fluid contains a plurality of rare cells and a plurality of other cells; (iii) incubating the immobilized antibody with the unit sample of bodily fluid under conditions suitable for binding of the antibody to the cell-surface antigen of the rare cell so as to form antibody-rare cell complexes; (iv) washing the antibody-rare cell complexes to remove the unbound cells and provide immobilized antibody-rare cell complexes; and (v) determining the number of immobilized antibody-rare cell complexes in the sample and thereby estimating the number of rare cells per unit of the sample fluid.

### BRIEF DESCRIPTION OF THE FIGURES

The application file contains at least one drawing executed in color. Copies of this patent or patent application publication with color drawing(s) will be provided by the Office upon request and payment of the necessary fee.

FIG. 1: Increasing concentrations of biotinylated 4B9 antibody were incubated under identical conditions for 30 mins with streptavidin coated magnetic particles (from Invitrogen Biotin binder, CELLECTIN, and FlowComp kits), dextran-coated nanoparticles (provided in StemCell technologies EasySep human biotin positive cell selection kit), and streptavidin coated microwells (Microwell-SA). After washing, bound 4B9 was detected and quantified colorimetrically using HRPO labeled goat anti-mouse IgM antibody.

FIG. 2: The isolated cells were fixed, permeabilized, and probed with AMCA-labeled mouse anti-human ε-globulin antibody. Representative images shown were acquired microscopically under bright field (BF), fluorescence field detecting ε-globulin positive responses, and the composite merged image.

FIG. 3: The isolated cells were fixed, permeabilized, and probed with AMCA-labeled mouse anti-human epsilon globulin antibody. Representative images shown acquired microscopically under bright field (BF), fluorescence field detecting ε-globulin positive responses, and the composite merged image.

FIG. 4: The isolated cells were fixed, permeabilized, and probed with AMCA-labeled mouse anti-human epsilon globulin antibody. The cells were then counter stained with TO-PRO. Representative images shown acquired microscopically under bright field (BF), fluorescence field showing nuclear and ε-globulin positive responses, and the composite merged image.

FIG. 5: Fetal NRBC were isolated from maternal blood (5 mL) of a confirmed 30 weeks gestation male pregnancy using 4B9(O)-coated glass slide. Isolated cells were probed for Y-chromosome (red) and X-chromosome (green), with composite merged image also shown.

FIG. 6: Is an Enlarged composite of the FISH image shown in FIG. 5.

### DETAILED DESCRIPTION OF THE INVENTION

Given the well known value of circulating fetal cells as complete source of fetal genetic material, there still remains an urgent need for their reliable and consistent isolation from maternal blood with high sensitivity and specificity. The present invention provides a two-site “sandwich-type” rare cell isolation technology, protocols, and platforms comprising pair-wise combinations of one or more cell capture antibody with one-or more antibodies for cell detection/identification. In certain embodiments the sandwich-type cell isolation and analysis technology of the present invention employs combinations of specific capture with non-specific detection, combinations of non-specific capture with specific detection, or any other suitable combinations that will be immediately recognized by those skilled in the art. This novel, highly efficient, and reliable technology can be easily configured into standalone manual rare cell isolation and analysis kits or adapted to automated applications compatible with routine laboratory use. Accordingly, in one embodiment the invention provides a simple, fast, reliable, and cost effective technology for a seamless single-step process of capture, isolation, and detection (and identification) of fetal nucleated red blood cells (NRBC) from maternal blood, and utility for non-invasive prenatal diagnosis of fetal genetic abnormalities.

In one embodiment of the methods of the present invention, mouse monoclonal antibody (antibody 4B9) specific for epitopes expressed on plasma membrane of fetal NRBC is coated onto a large-surfaced solid support. In certain embodiments of the present invention the solid support can be colloidal metal particles (such as colloidal gold particles), magnetic particles (such as ferrous metal particles), a magnetic plate, magnetic jackets, magnetic rods, polymeric beads, surfaces of medical and mechanical micro devices, surfaces of medical and mechanical microelectronic devices, and surfaces of medical and mechanical microelectronic sensors. Detection and/or identification of the specifically captured fetal NRBC can be accomplished using 4B9 antibody labelled with a reporter molecule. Alternatively, 4B9 or one or more antibodies of similar specificity can be used in any possible sandwich combinations with one or more antibodies against known or yet to be discovered cell surface and/or internal fetal NRBC identifying biomarkers. For example, 4B9 or other anti-fetal NRBC antibodies can be combined as capture or detection antibodies with one or more specific or non-specific fetal NRBC detection antibody. Such combinations include detection of 4B9-captured fetal NRBC by appropriately labelled antibody against specific (e.g., fetal epsilon globulin) and/or non-specific (e.g. cell surface glycophorin-A, and/or i-antigen) fetal NRBC biomarkers. Possible fetal NRBC capture/detection antibody combinations include 4B9/anti-CD36; 4B9/anti-CD71; 4B9/anti-CD 47; anti-CD36/4B9; anti-CD71/4B9; anti-CD47/4B9; anti-CD36/anti-CD47; anti-CD36/anti-CD 71; anti-CD36/anti-glycophorin-A; anti-CD36/anti-1-antigen. Fetal NRBC detection/differentiation can also include nuclear stains and can be expanded to include other suitable sandwich combinations of antibodies against other readily available fetal NRBC differentiating biomarkers.

The invention provides a single-step, continuous, and seamless reliable method for detection, isolation and analysis of circulating rare cells of interest from biological sources, such as, for instance, circulating fetal nucleated RBCs from maternal blood. Other examples of rare cells that can be isolated from biological fluids by methods of the present invention include cytotrophoblast cells that can be isolated from a suspension of cells obtained from biopsy samples of chorionic villus sampling (CVS); amniocytes from amniotic fluid obtained by amniocentesis; and leukocytes from urine samples, such as from patients suffering from diseases and conditions e.g. urinary tract infections.

As used herein, a rare cell is a cell that has at least one characteristic cellular antigen that is not present in the majority of the cellular population in which it is found. Alternatively, the rare cell can have a characteristic antigen that is different from the homologous antigen in the majority of cells of the cellular population in which it is found. For instance, characteristic cellular antigen of the rare cell can be a cell surface antigen, a cytoplasmic antigen or a nuclear antigen. The characteristic antigen of the rare cell can be an antigen of a cellular component not found in the majority of the cellular population, or it can be an antigenic variant of a cellular component found in the cells of the majority of the cellular population. For example, the NRBC antigen bound by antibody 4B9 is not present on mature red blood cells of non-pregnant adults.

The rare cell can be a cancer cell, such as for instance a tumor cell, an adenoma cell, a carcinoma cell or any other cancer cell. The rare cancer cell can be a circulating tumor cell in a blood sample, or a rare cancer cell in a population of normal cells in a biological fluid; the biological fluid can be any biological fluid including, but not limited to a suspension of cells originating from a tissue biopsy.

The rare cell can represent one cell in from about 10² to about 10⁴ cells, from about 10³ to about 10⁵ cells, from about 10⁴ to about 10⁶ cells, from about 10⁵ to about 10⁷ cells, from about 10⁶ to about 10⁸ cells, from about 10⁷ to about 10⁹ cells, or even from about 10⁸ to about 10¹⁰ cells of a cell population in a biological fluid. The biological fluid can be any biological fluid, such as for instance and without limitation, blood, plasma, or urine; or the biological fluid can be a suspension of cells obtained from a tissue sample, such as a biopsy sample.

As used herein a mammal can be any mammal, such as for instance and without limitation, a human or an animal; the animal can be any animal, such as a non-human primate e.g. a chimpanzee, a gorilla or an orangutan; the animal can be a companion animal e.g. a dog or a cat; alternatively, the animal can be a farm animal such as a cow, a sheep, a pig or a goat; the animal can also be a zoo animal such as a bear, a tiger, or a lion.

In one embodiment of the methods of the present invention, isolation of fetal NRBC specifically involves a short (e.g. 30-60 minutes) incubation of maternal blood (5-10 mL) with a cell isolation substrate coated with 4B9 antibody. After washing to remove unbound cells, the immobilized fetal NRBC is incubated for 30-60 minutes with 4B9 antibody labelled with a suitable detection moiety. Because of high specificity of 4B9 antibody for fetal NRBC, the high isolation efficiency of the strategy, and the implemented washing step, the combined detection/identification of the isolated cells can be readily achieved by using labelled 4B9 or a suitable labelled antibody against other specific or non-specific NRBC identifiers described above. In addition to allowing for combined fetal NRBC capture, detection, and identification, the technology is also compatible with the intended analysis procedures directly on the cells bound to the isolation substrate, using appropriate and readily available chromosomal, genetic, and molecular tests known to those of skill in the art.

Alternatively, the high purity and large numbers of the isolated cells provide for easy access to single fetal NRBC for micromanipulation or scraping the entire population of captured fetal NRBC from the solid-phase substrate for downstream genetic and molecular testing. This novel sandwich-type cell capture, detection, identification technology can be readily used for general application to isolation of any rare cell population from human or animal biological fluids, such as blood, amniotic fluid and urine; and also for isolation of any rare cell population from a suspension of human or animal cells from a biopsy. The specifically isolated cells can be used for research, for evaluation of cell responses to pharmaceutical agents, or for indication of diseases such as chromosomal and genetic abnormalities, maternal complications of pregnancy, and various cancers to name a few. The only requirement is the availability and/or development of antibodies that selectively or specifically bind to the intended target cell. An additional adaptation of the present invention is its application as a diagnostic method based on monitoring changes in circulating numbers of rare cells such as fetal NRBC in relation to occurrences of fetal and/or maternal complications. There are reportedly more fetal cells entering maternal blood in conditions such as Down syndrome (DS) and preeclampsia. Preeclampsia is a pregnancy condition in which high blood pressure and protein in the urine develop after the 20th week (late second or third trimester) of pregnancy. In such conditions, comparative analysis of relative changes in the number of isolated fetal NRBC per unit of maternal blood obtained from suspected vs. gestation-matched normal pregnancies is useful for diagnosis and is also of value in predicting onset of these conditions.

Pair-wise combinations of antibodies that react with specific and/or non-specific fetal NRBC surface antigens in a two-site “sandwich-type” approach is an important design component of the present invention. Until now, the state of the art in fetal cell isolation has generally focused on multi-steps cell enrichment approaches that are relatively complex, have insufficient sensitivity, and are prone to poor yield and/or significant cell loss and give inconsistent results. In addition, reported approaches generally target fetal cell markers that are non-specific and/or subject to altered expression as target cells undergo maturation processes (17, 18, 20-28).

In one embodiment, the present invention incorporates the specific fetal NRBC recognition property of a new monoclonal antibody (antibody 4B9 described in U.S. Pat. No. 7,858,757 B2) combined with a two-site “sandwich-type” design providing a reliable method for highly efficient and convenient isolation of fetal NRBC from maternal blood. In this novel design, 4B9 antibody, recognizing a specific cell surface epitope, is coated onto a suitable reaction surface and the specifically captured fetal NRBC are detected using 4B9 antibody covalently or non-covalently coupled to a readily quantifiable/detectable label. Because of the intrinsic flexibility of the sandwich-type cell isolation approach, allowing for sequential process of cell capture, cell wash to remove unbound cells, and cell detection, a specific capture antibody such as 4B9 can be alternatively paired with one or more detection antibody against specific (example; anti-epsilon globulin) or non-specific (example; glycophorin-A, i-protein, CD47) fetal NRBC identifiers.

Combinations of capture/detection antibodies that either bind to the same or different fetal NRBC surface antigens, such as 4B9/4B9 or 4B9/anti-glycophorin-A antibody, add another novel dimension of specificity and accuracy to the technology of the present invention. The cell capture/detection strategy provided is not limited to pair-wise antibody combinations and can be readily configured to include one or more capture antibodies in combinations with one or more detection antibody against internal and/or external fetal NRBC identifiers known to those skilled in the art.

The use of antibody-coated large surfaced flat or contained solid-supports such as the readily available microscope slide and Petri-dish has several advantages. In addition to facilitating closer contact and providing for increased cell capture capacity and affinity independent reaction kinetics (30), they allow for unification of the various required steps into a simple and continuous process that serve to minimize errors and increase consistency. This single format system is highly advantageous as the methods of the present invention combine the steps of cell capture, washing to remove unbound cells, and cell detection (identification) as well as analysis into a seamless platform system suitable to both manual and automated applications.

This unified process has recognizable operational benefits as multi-step approaches requiring different formats for cell enrichment, identification and/or isolation are prone to cumulative errors and cell loss, thus making development of consistent cell isolation methods with high efficiency difficult if not impossible (18). The present invention can be offered as a standalone antibody-based fetal NRBC isolation kit for general downstream use, or be provided as a complete fetal NRBC isolation and analysis kit. The flexibility of design, allowing integration of cell isolation platform with antibodies of different specificity in a sandwich-type cell capture/detection approach provides broad applicability of the present invention to isolation and analysis of any circulating rare cell of research and/or clinical interest.

### Materials and Methods

### Patient Population and Sample

Peripheral blood was collected from first trimester pregnancies between 8 to 12 weeks of gestation (age 22-45) and from ultrasound confirmed second trimester male pregnancies. Blood samples were also collected from nonpregnant women. Samples from pregnant and non-pregnant women were obtained from Dr. Jonathan Herman, Long Island Jewish Medical Centre, NY. Specimens from male subjects were obtained from volunteering staff at KellBenx, Great River, N.Y. All specimens were collected in EDTA containing blood collection tubes after obtaining informed written consent from blood donors. All blood samples were used within 24 hours of collection.

### Materials

Horseradish peroxidase (HRP) and streptavidin were obtained from Scripps Laboratories, San Diego, Calif. Sulfo-NHS-LC-LC biotin, Sulfo-NHS-SS-Biotin, NHS-PG12-Biotin, and NHS-SS-PG12-Biotin; disulfide bond breakers, Dithiotheritol (DTT), and TCEP Solution; Goat anti-mouse IGM(u), Rabbit anti-mouse IGM(u), and Goat anti-Mouse IGM, Fab₂; FC receptor blocker were obtained from ThermoFisher Scientific (www.Thermofisher.com). EPS Microarray microscope glass slides, Superfrost Gold microscope glass slides, Screw cap slide holders; Fisher brand 100 mm and 60 mm Petri dish, and Flat bottom 6 well non-tissue culture plates were from ThermoFisher.

Dynabead® biotin binder magnetic beads coated with streptavidin; CELLectin Biotin Binder kit, involving magnetic beads coated with streptavidin via a DNA linker to provide a DNase cleavable site for release of cells bound to a biotinylated anti-cell antibody; and Dynabeads® FlowComp Flexi, Part A and Part B, kit involving magnetic particle coated with modified streptavidin, a DSB-X biotin antibody labelling kit, and a D-biotin-based releasing agent for release of cells bound to a DSB-X biotinylated anti-cell antibody were obtained from Invitrogen (www.invtrogen.com). Heat inactivated fetal bovine serum, RPMI medium 1640, D-PBS without calcium or magnesium, and purified mouse IgM were from Invitrogen.

EasySep® human biotin positive cell selection kit, involving dextran-coated magnetic nanoparticles using bispecific tetrameric antibody complex (TAC), that recognizes both dextran and the biotin molecule attached to the anti-cell antibody was obtained from StemCell Technologies (www.stemcell.com). SuperEpoxy® glass slides were obtained from Arrayit Corporation (Sunnyvale, Calif. 94089). Surface activated Nexterion® glass slide H and P were obtained from SCHOTT North America Inc., Louisville, Ky. 40228.

Mouse IgG solution, Mouse serum, Goat IgG solution, and Goat serum were obtained from Equitech-Bio, Inc., Kerrville, Tex. 78028. Tetramethylbenzidine (TMB) microwell peroxidase substrate system was from Neogen Corporation, Lexington Ky. FITC (fluorescein isothiocyanate), AMCA (7-amino-4-methylcoumarin-3-acetic acid), Alexa Fluor® 350, and DyLight350 were from Invitrogen, and Thermo Scientific. TO-Pro for nuclei staining was obtained from Invitrogen. Commercial antibodies against CD36, CD71, and glycophorin-A were from Invitrogen. Antibody recognizing fetal epsilon globulin was from Fitzgerald Industries International (www.Fitzgeral-fii.com). Antibodies purchased pre-labelled with the detection probe or labelled in-house using manufacturer's instructions.

Reagents and kit for performing FISH (fluorescence in situ hybridization) were form AneuVysion (www.abbottmolecular.com). All other chemical reagents were of highest quality and were obtained from Sigma Chemical Co., St. Louis, Mo., or Amresco, Inc., Solon, Ohio. Eight well microtitration (microwells) strips and frames were products of Griner International, Germany.

### Antibodies

The hybridoma clone that secretes Monoclonal antibody 4B9 has been deposited with the Deutsche Sammlung von Mikroorganismen and Zellkulturen GmbH (DSMZ, Braunschweig, Germany) under the accession number DSM ACC 2666.

Anti-fetal NRBC antibodies may be monoclonal, polyclonal or any other fetal NRBC binder combinations. Suitable two-site “sandwich-type” cell capture and detection and/or identification binding partners with broad or exclusive binding affinity for various surface and/or internal antigenic determinant expressed by rare cells such as fetal NRBC can be used in the methods of the present invention. These can be also based on pair-wise selection of commercially available antibodies and reported expression and specificity. For example, the list of commercially available and proprietary antibodies that recognize fetal NRBC includes but is not limited to antibodies reacting with cluster of cell surface differentiation markers (CD) such as CD36, CD71, CD47, as well as antibodies against glycophorin-A, i-antigen, and ε-globulin.

The method for preparation of monoclonal as well as polyclonal antibodies is now well established [Harlow E. et al., 1988 Antibodies. New York, Cold Spring Harbor Laboratory]. Monoclonal antibodies can be prepared according to the well established standard laboratory procedures “Practice and Theory of Enzyme Immunoassays” by P. Tijssen (In Laboratory Techniques in Biochemistry and Molecular Biology, Eds: R. H. Burdon and P. H. van Kinppenberg; Elsevier Publishers Biomedical Division, 1985), which are based on the original technique of Kohler and Milstein (Kohler G., Milstein C. Nature 256:495, 1975). Antibodies can also be produced by other approaches known in the art, including but not limited to immunization with specific DNA.

A particular consideration for pair-wise antibody selection is the ability of the capture antibody, coated onto a solid-support, and the detection antibody, conjugated to a detection label, to bind simultaneously to the same or different determinants expressed on the surface of fetal NRBC differentiating biomarkers. The fetal NRBC capture/detection binding partners can also include antibody fragments, chimeric antibodies, humanized antibodies, antibody and cell binding peptides developed by re-engineering of existing antibodies, synthetic antibodies, synthetic binders, recombinant antibodies as well as peptide and protein binders selected by screening phage display libraries and other similar expression and selection systems.

Polyclonal or monoclonal antibodies can be raised by standard well known methods against whole fetal NRBC, against fetal NRBC sub-fractions such as isolated cell membranes, isolated nucleus and isolated plasma membrane; against fetal NRBC progenitor and/or fetal stem cells; against fetal cell soluble proteins, peptides, and glycoprotein; against other relevant antigenic molecules and known or yet to be discovered structures. Other suitable antigens for immunization include, but are not limited to synthetic peptides, designer molecules, and fetal NRBC antigen-mimicking structures. Antibodies can be raised in various species including but not limited to mouse, rat, rabbit, goat, sheep, donkey, horse and chicken using standard immunization and bleeding procedures. Animal bleeds or hybridoma cell culture media can be fractionated and purified by the well established and widely available standard antibody purification schemes.

### EXAMPLES

### Cell-Free 4B9 ELISA

The 4B9 ELISA of one embodiment of the present invention involves direct or indirect coating of 4B9 antibody onto solid-supports, detecting bound 4B9 using goat ant-mouse IgM (Fab)₂ labelled with the enzyme horseradish peroxidase (HRPO), and colorimetric quantification of the reaction using HRPO substrate Tetramethylbenzidine (TMB). Whereas in direct coating, 4B9 was detected by incubation with the detection anti-mouse antibody-HRPO conjugate (0.025 ug/mL of assay buffer; 10 mM NaPO₄, pH 7.4, containing 8.8 g NaCl, 0.5 g BSA, 0.5 mL Tween-20, and 2.5 mL proclin per litre), the indirect coating involved pre-incubation of unlabeled or biotinylated 4B9 (10 ug/mL) with second antibody or streptavidin coated support, respectively.

In general, comparative evaluation of microtitration wells, magnetic particles in test tubes, and glass slides in 16-well partitioned assemblies (Grace Bio labs) were performed under nearly identical conditions of antibody volume (50 uL/reaction), assay buffer volume (100 uL/reaction), and 60 min shaking or mixing incubation. After four times wash with ELISA wash buffer (0.05 mM Tris, pH 7.4, containing 0.05% Tween-20) and addition of the detection antibody-HRPO conjugate (100 uL/assay), each reaction support was washed as above and incubated for 10 minutes with 100 uL of TMB substrate. In the case of magnetic particles and partitioned glass slides, 100 uL of reacted substrate was then transferred into clean microtitration wells. This was followed by addition of 100 uL/well of stopping solution (0.2 M Sulphuric acid) to all wells and comparative dual wavelength absorbance measurement at 450 and 620 nm. Optimization and evaluation of antibody coated onto Petri-dish or six well tissue culture plates was as above, except larger volumes of the various reagents were used.

General procedures for coating antibodies or streptavidin onto microtitration wells or other supports were as previously described (31-34). Magnetic particles obtained commercially were mostly coated with streptavidin or anti-mouse IgM. The beads (25 uL containing 1×10⁷ beads) were washed as per manufacturer instructions and incubated with increasing concentrations of biotinylated 4B9 or unlabeled 4B9 antibody. Bead-bound antibody was resuspended in 100 uL of the assay buffer and incubated with 100 uL of the anti-mouse IgM-HRPO conjugate and the reaction was quantified as described above. Commercial glass slides that had been functionalized for covalent or non-covalent protein labelling were coupled with antibody or streptavidin according to previously published methods (31-34) or manufacturer's instructions. For labelling the entire activated surface, the slides were secured into one-well slide assembly (Grace Bio-lab) and incubated with 4-mL/slide of the coating antibody or streptavidin. For comparative testing, the slides were partitioned into 16-well assemblies and to each well added 100 uL of increasing concentrations of biotinylated 4B9 or unlabeled 4B9 antibody. After incubation and washing, 100 uL/well of the anti-mouse IgM-HRPO conjugate was added and the reaction quantified as described above. Antibody or streptavidin coating onto Petri-dish or six well tissue culture plates was as above, using appropriate volumes of the coating and blocking buffers. For comparative evaluations, the widely used clear eight well-strip plates (Griner Bio-One, Microclon 600 high binding), coated with streptavidin or 2nd antibody, were similar assayed for binding to 4B9 as described for magnetic particles and glass slides.

Protocols for coupling of the detection antibody to HRPO was performed as described (31-34). The coupling reaction involved activation of the enzyme with Sulfo-SMCC and its subsequent reaction with the detection anti-mouse antibody, which had been activated by 2-iminothiolane. Coupling of antibodies to biotin was performed according to standard procedures (34).

This simple and quantitative ELISA system was subjected to comparative evaluation of 4B9 binding characteristics and (a) widely used liquid-phase magnetic particles, (b) widely used microtitration wells, and (c) to large-surfaced solid-phase supports such as microscope slides, Petri-dish, and large six-well tissue culture plates. The ELISA facilitated rapid development, optimization, and comparative evaluations of numerous aspects important to evolution of the disclosed cell isolation technology and platform, which would have been otherwise extremely difficult if not impossible to ascertain. The latter included but is not limited to (1), comparative assessment of non-covalent (passive) or covalent binding properties of 4B9 at various concentrations (0.5-40 ug/mL) and in various coating buffers (phosphate, pH 6.5, phosphate, pH 8.0, borate, pH 8.5, carbonate, pH 9.1) to various supports (2), non-covalent binding properties and 4B9 binding capacity of anti-species antibodies (e.g., goat anti-mouse IgM) coated at various concentrations (1-40 ug/ml), in above buffers, to various supports (3), binding of increasing amounts of 4B9 antibody labelled with five different biotin-labelling agents (see materials) in various molar ratios (10-400 mole biotin/mole antibody) to various commercial and/or in-house manufactured streptavidin coated supports and (4), binding of increasing amounts unlabeled 4B9 to optimally coated second antibody (e.g., goat anti-mouse IgM) to various supports.

### Cell Capture

Capture antibodies can be non-covalently coated on, covalently coupled with, or linked to various solid phase supports using standard non-covalent or covalent binding methods. The solid support can be in the form of test tube, beads, microparticles, filter paper, various membranes, glass filters, glass slides, glass or silicon chips, magnetic nano- and microparticles, magnetic rods, magnetic sleeves as well as microfluidic, microelectronic, and micromagnetic mechanical cell separation systems and devices, various glass or plastic chambers, or other materials and supports known in the art. The latter can also include various medical devices for insertion into patient circulation for in-vivo collection of cells.

### Cell Release

Supermagnetic micro- and nano-particles coated with specific antibodies, with avidin, streptavidin, or their modifications, or with anti-species antibodies as well as with affinity binders such as protein-A or protein-G have dominated the field of cell isolation. These approaches generally involve magnetically labelling antibodies of desired specificity, incubating the magnetized antibody with target sample (e.g., maternal blood), and retaining target cells (positive selection) or unwanted cells (negative selection) when a strong magnet is placed outside the incubating chamber (18).

Although the immunomagnetic cell sorting (MACS) methods are relatively convenient, inexpensive, and easy to operate, the technology is reported prone to several significant limitations including inefficiency, poor yield, cell entrapments, bead-to-bead interaction or aggregations, cell damage, inconsistency, and autofluoresence interferences with immunostaining methods. To improve performance, several sample pre-treatment enrichment methods (filtration, density gradient separation, differential cell lysis or sized based separation with or without negative immunoselection) that are also prone to significant errors, cell loss, and inconsistency have been employed (18, 23, 29).

Strategies have been developed to dissociate the capture cells from magnetic particles by incorporating a cleavable linkage between the particles and the employed antibody. Alternative strategies involving displaceable biotin labels or antibody detachment by competing antibodies have also been developed and are commercially available (Invitrogen; Miltenyi Biotech; Stem cell technologies).

Antibody 4B9 was used in association with cleavable (disulfide-linked) biotin (e.g., Sulfo-NHS-SS and NHS-SS-PG12-Biotin), DXB-X-biotin included in the Invitrogen Biotin binder kit, and in association with the Invitrogen CELLectin kit. After coupling 4B9 to streptavidin-coated wells or magnetic particles provided in corresponding kits, the solid-support-4B9 complexes were incubated for 30 minutes with increasing concentrations of the cleaving or displacing agent. Solid supports were then washed and the reaction developed using Cell-Free 4B9 ELISA described above. The efficiency of the antibody releasing system was readily determined by comparing signals remaining in the treated tests vs. total signal generated in untreated control tests.

### Detection and Identification of Captured Cells

Antibody used to detect captured cells can serve the dual purpose of cell detection as well as identification. The latter is possible particularly by use of two-step immunoreaction protocols (31-35) in which capture of target molecule by a specific antibody is followed by a washing step, to remove unattached molecules, and detection of specifically captured molecule by a specific and/or non-specific detection antibody. The fact that in a two-step capture/detection format, specifically captured blood molecules can be detected by non-specific or broadly reactive detection antibodies (36), is further testament of advantage and flexibility of the methods of the present invention as specifically captured cells can also be accurately detected using a non-specific cell detection antibody.

Application of the above concept to fetal NRBC isolation was explored by capturing fetal cells from maternal blood, washing to remove unattached cells, and detecting captured cells with a specific and/or non-specific detection antibody. In a series of parallel two-step “sandwich-type” experiments, fetal NRBC captured by solid-phase 4B9 antibody were, after washing, detected by labelled 4B9 or by another labelled antibody broadly recognizing surface markers expressed on various fetal and even maternal blood cells (e.g., antibody reactive with GPH-A, CD36, CD71, or CD47). After a second washing step, the isolated cells were also stained for epsilon globulin and analyzed microscopically. In all cases, isolated cells stained with the detection antibody were also stain for epsilon globulin, confirming the specificity of the technology and identity of the specifically captured/detected cells as fetal primitive NRBC. Because of specificity epsilon globulin for primitive fetal cells only (17, 20, 22) and specificity of 4B9 for both primitive and definitive cells, it is possible to detect fetal cells not stained for epsilon globulin. However, primitive NRBCs are the predominant cell types in first trimester maternal blood until 12 weeks gestation (20).

The concordance of surface staining of 4B9-captured fetal cells by the detection antibody and cytoplasmic staining by epsilon globulin antibody has significant implications. This observation for the first time demonstrates and confirms that it is possible to efficiently capture, detect, and identify circulating rare cells using a simple two-step sandwich-type method to provide a highly sensitive, specific, and reproducible immunoassay for quantification of circulating blood molecules.

The detection antibody can be either directly coupled to a reporter molecule, or detected indirectly by a secondary detection system. The latter may be based on any one or a combination of several different principles including but not limited to antibody labelled anti-species antibody and other forms of immunological or non-immunological bridging and signal amplification systems (e.g., biotin-streptavidin technology, protein-A and protein-G mediated technology, or nucleic acid probe/anti-nucleic acid probes and the like). The label used for direct or indirect antibody coupling may be any detectable reported molecule. Suitable reporter molecules may be those known in the field of immunocytochemistry, molecular biology, light, fluorescence, and electron microscopy, cell immunophenotyping, cell sorting, flow cytometry, cell visualization, detection, enumeration, and/or signal output quantification known to those skilled in the art.

Examples of suitable labels include, but are not limited to fluorophores, luminescent labels, metal complexes, radioisotopes, biotin, streptavidin, enzymes, or other detection labels and combination of labels such as enzymes and a luminogenic substrate. Example of suitable enzymes and their substrates include alkaline phosphatase, horseradish peroxidase, beta-galactosidase, and luciferase, and other detection systems known in the art. More than one antibody of specific and/or non-specific nature might be labelled and used simultaneously or sequentially to enhance cell detection, identification, and/or specificity. In such application, each antibody is labelled with different label known in the art of having different and differentiating signal output property, detection signal, spectra, or fluorescent emission spectra. Example of suitable labels widely used in the field of immunocytochemistry and cell detection microscopy include, but are not limited to FITC (fluorescein isothiocyanate) AMCA (7-amino-4-methylcoumarin-3-acetic acid), Alexa Fluor 488, Alexa Fluor 594, Alexa Fluor 350, DyLight350, phycoerythrin, allophycocyanin. Stains for detecting nuclei include Hoechst 33342, LDS751, TO-PRO and DAPI.

### Fetal NRBC Isolation Immunoassay

The fetal NRBC isolation assay according to one embodiment of the present invention provides a two-site “sandwich-type” immunoassay, performed in a two-step “sequential” process of a first incubation step, washing, and a second incubation step. In the assay, an appropriate volume of washed whole blood was added to directly or indirectly (via Streptavidin or second antibody) 4B9 pre-coated dish (10 mL/dish), six-well tissue culture plates (3 mL/well), or glass slide (10 mL/2 slides in plastic slide containers) and incubated for 60 min with continuous gentle mixing. After incubation, blood was removed by gentle aspiration, and the incubating chambers or slides were washed five times with appropriate volume of PBS (GIBCO DPBS). This was then followed by incubation as above with appropriately diluted detection antibody 4B9 or any other appropriately labelled fetal NRBC identifying detection antibody. After incubation and washing as above, the isolated cells are ready for further processing. Examples of such processing include but are not limited to fixing, permeabilizing, and immunoprobing for fetal NRBC indentifying markers such as epsilon globulin as well as nuclear counterstaining according to established and reported procedures (20, 22, 23). Alternatively, the cells can be subjected to chromosomal analysis by FISH (fluorescent in situ hybridization) for indication of specific chromosomal and genetic abnormality using established methods (21, 37) and commercially available reagents from suppliers e.g. AneuVysion (www.abbottmolecular.com).

The cells can be also removed by micromanipulation or by scraping the entire cell population from the substrate or support for downstream chromosomal, molecular, and gene sequencing technologies according to readily available and well known methods. The latter include but are not limited to FISH for aneuploidies (21, 18, 13, X, and X), QF-PCR for aneuploidies, Array-CGH, or genome sequencing for genetic mutations or polymorphisms using the widely available commercial reagents, kits, and instrumentations from several commercial companies. Examples include BioReference Laboratories, Abbott's Aneuvysion, GenomeDX, Gen-Probe, Signature Labs, Ambry Genetics, Invitrogen, Beckman, Bio-Rad, Molecular devices, Applied Biosciences and Illumina Inc.

Whole blood (maternal blood, non-pregnancy bleed, male blood) collected in EDTA tubes were centrifuged at 2000 rpm (Beckman Allegra) for 10 minutes. The plasma fraction was discarded and the cell layer resuspend, at 1+2 volume ratios, in buffer #1 (Ca²⁺ and Mg²⁺ free PBS with 0.1% BSA, 5 mM EDTA, 2.5% FC receptor blocker), mixed gently, and centrifuged as described above. The cell layer was washed twice again and resuspended to original blood volume prior to use.

Solid supports were coated with antibody or protein at 10 ug/mL of coating buffer (50 mM Sodium Borate, pH 8.5) using published methods (31). In brief, supports were incubated with an appropriate volume of the coating capture antibody or protein solution overnight at room temperature. Coated supports were then washed once with support wash buffer (10 mM KPO4, pH 7.4) and incubated for 1 hr in appropriate volume of the blocking solution (wash buffer containing 1% BSA). The solid-supports were washed once with the wash buffer prior to use or stored at 4° C. for up to 1 week in the blocking buffer. Antibodies or proteins can be coated onto the various supports and provided in a ready to use dry format. Coupling of detection 4B9 antibody and other suitable detection and/or confirmatory antibodies to various fluorescent probes (example FICT) can be readily performed using reagents and kits available from several commercial companies such as Invitrogen (www.invitrogen.com) and Thermo Scientific (www.piercenet.com).

### Cell Staining and Analysis Methods

Methods for immunofluorescence, immunoenzymatic, and cytochemical staining of cell membrane, cytoplasm, and organelles are now well established and widely available commercially. This includes fixing, permeabilizing, and probing for fetal NRBC indentifying markers such as CD antigens, GPH-A, 1-antigen, and fetal epsilon globulin as well as nuclear counterstaining according to established and reported procedures (20, 22, 23). Technologies for chromosomal staining by FISH are well established (21, 37) and commercial reagents and kids widely available [AneuVysion (www.abbottmolecular.com)]. Technologies for downstream genetic and/or molecular testing are also widely available. The latter include but not limited to QF-PCR for aneuploidies, Array-CGH, or genome sequencing for all genetic malformations using the widely available commercial reagents, kits, and instrumentation.

In one embodiment of the present invention, 4B9 captured cells were stained for fetal epsilon globulin using a monoclonal antibody labelled with DyLight350 or Alexa Fluor 350 according to established methods. In brief, after completing the washing step, the capture cells were fixed with cold methanol (−20° C.) for 10 min and with 4% formalin for 10 min at room temperature. After washing, cells were permeabilized) with 0.1% Triton X-100 in PBS (5 min at room temperature), blocked with 1% BSA in PBS and incubated with labelled anti-epsilon globulin antibody in the same buffer (2 hrs at room temperature or overnight at 4° C.). For counterstaining of cell nuclei, appropriate volume of a 4 uM solution of TO-PRO-1 in PBS was added, the incubating reaction covered with foil, and incubated for 10 min at room temperature. Cells where then washed once with PBS prior to analysis. Chromosomal FISH was done as per instruction of manufacturer reagents and kits (AneuVysion, see www.abbottmolecular.com).

### Data Analysis

Colorimetric ELISA results were analyzed using the data reduction packages included in the Labsystems Multiskan microplate ELISA reader (Labsystems, Helsinki, Finland). Cell images were captured by microscopy (Nikon Eclipse 50i or Nikon Eclipse TI-S) using QI-CLICK monochrome camera and NIS Elements software. Enumeration of isolated cells was done by manual scanning and recording. All plots and statistical analysis were performed by SigmaPlot® and SigmaStat® (Superior Performing Software Systems Inc, Chicago Ill. 60606-9653).

### Results

### Cell Free 4B9 ELISA

In the 4B9 ELISA, 4B9 antibody can be directly or indirectly (via streptavidin or anti-species antibody) coupled to various supports and the binding capacity and efficiency can be rapidly and quantitatively compared to existing and an antibody capture substrate such as microtitration wells.

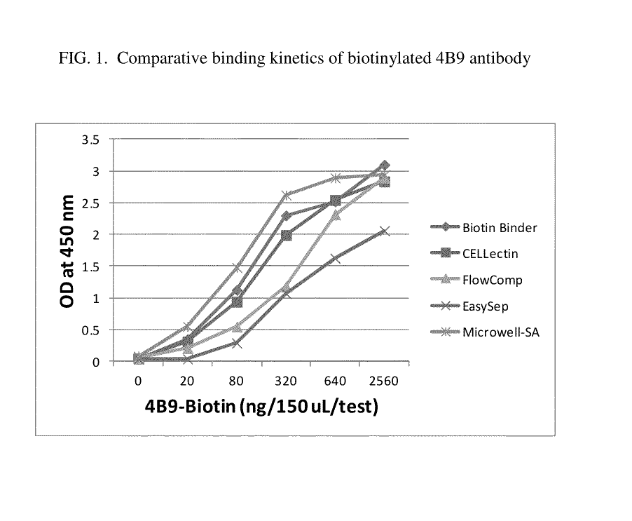

In one embodiment, the cell-free 4B9 ELISA employs a two-step noncompetitive immunoreaction in which covalent or non-covalent binding of 4B9, streptavidin, or second antibody to supports was comparatively and colorimetrically quantified by interaction of bound 4B9 with the detection goat anti-mouse IgM labeled with HRPO. The optimized protocol was established by investigating the effects of various parameters and technical manipulations on analytical performance (31-36). The best performance obtained with coating antibody or protein concentration of 5-10 ug/mL, detection antibody concentration of 0.2-0.5 ug/mL, and 30-60 min room temperature incubations, depending on whether direct or indirect (streptavidin or second antibody) coating systems were being evaluated. After washing, and 10 min incubation with TMB substrate, the reaction was stopped by addition of equivalent volume of the stopping solution followed by absorbance readings at 450 nm. Representative results of parallel evaluation of streptavidin-coated liquid-phase magnetic particles vs. solid-phase microtitration wells for their relative effectiveness in binding biotinylated 4B9 antibody is depicted in FIG. 1. Surprisingly, and in contrast to theoretical expectations (29), solid-phase microwells showed consistently better 4B9 binding kinetics and capacity. Results were similar when 4B9 was coated directly or via second antibody interface to comparative liquid-phase vs. solid-phase supports (data not shown).

### Cell Capture Platforms and Application to Cell Isolation

Large-surfaced solid-supports (glass slides and Petri-dish) were coated directly with 4B9 antibody or via streptavidin (biotinylated 4B9) or second antibody (unlabeled 4B9) interface. The solid-phase supports were then comparatively evaluated for their efficiency in isolating fetal NRBC from first trimester maternal blood. In these trials, two sets of separate experiments were performed.

### Experiment #1

Maternal blood from four first trimester pregnancies (30 mL total volume) were washed, resuspended to original volume, and pooled. Equal volumes of pooled blood (10 mL) were added to each of three Petri dishes coated with 4B9 antibody old lot (PD#1; 4B9-O), 4B9 antibody new lot (PD#2; 4B9-N), or anti-mouse IgM coupled with 4B9-O antibody (PD#3). After 60 minutes incubation with gentle mixing on a flat orbital shaker, cells were washed 5× with PBS and stained for detection of fetal epsilon globulin. From this first trimester pooled blood, the total number of isolated fetal cells stained positively for epsilon globulin in PD#1, PD#2, and PD#3, were 909 (91/mL of blood), 1192 (119/mL of blood), and 580 (58/mL of blood), respectively (See Table 1).

TABLE 1
Isolation of Fetal NRBC from Pooled First Trimester Maternal Blood

| Solid | Capture | Pooled | Blood | Pretreatment/ | Hb detection | Fixed & | No. of Cells |
|---|---|---|---|---|---|---|---|
| Support | Ab | Blood | Vol/Test | RBC lysis | antibody | Permeabilized | Isolated |
| PD#1 | 4B9(O) | 1^{st} | 10 mL | no | AMCA | yes | 909 |
| | | Trimester | | | mAB human | | |
| | | | | | epsilon Hb | | |
| PD#2 | 4B9(N) | 1^{st} | 10 mL | no | AMCA | yes | 1192 |
| | | Trimester | | | mAB human | | |
| | | | | | epsilon Hb | | |
| PD#3 | 2nd-Ab- | 1^{st} | 10 mL | no | AMCA | yes | 580 |
| | 4B9(O) | Trimester | | | mAB human | | |
| | | | | | epsilon Hb | | |

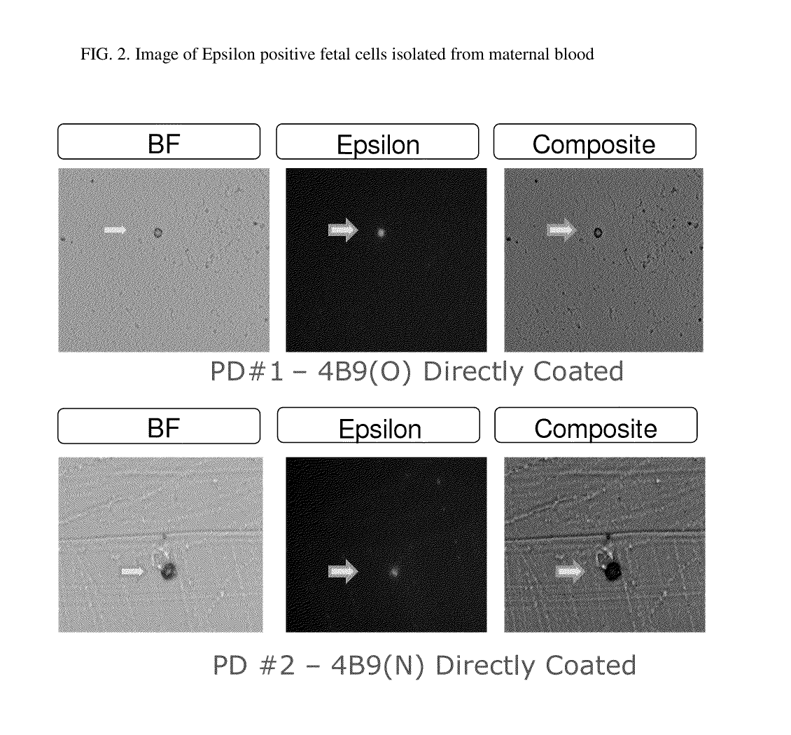

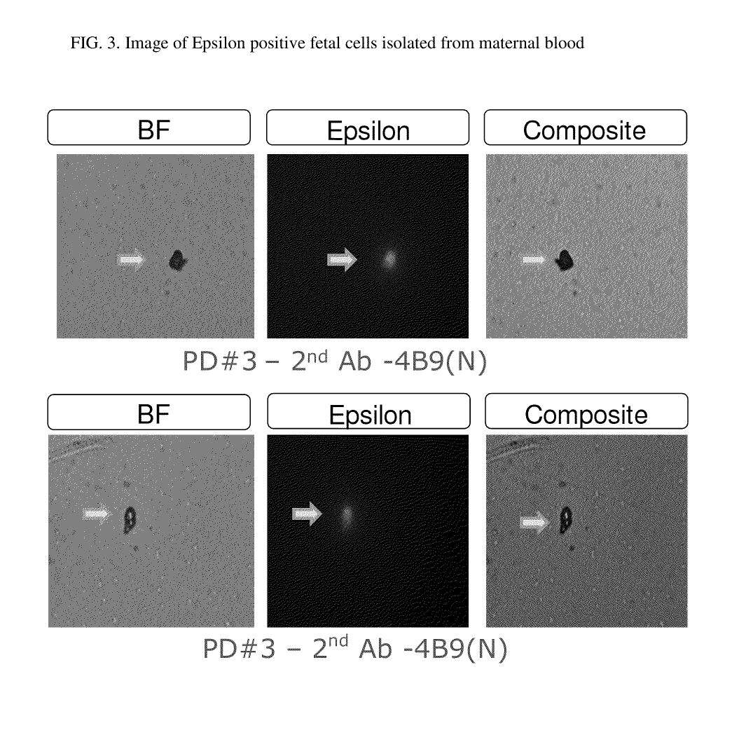

Representative images of epsilon-positive cells isolated by the various platforms are shown in FIG. 2, and FIG. 3. As epsilon globulin is reportedly a highly specific primitive fetal NRBC identifier (20, 22, 39), these findings disclose for the first time that circulating numbers of fetal cells in maternal blood are many fold higher than previously known, believed, or reported. The number of isolated fetal NRBC cells in the range of 60-120 cells per mL of maternal blood is an unprecedented discovery as the previously reported numbers are generally in the range of 1-2 cells/mL (18, 21-23, 27-29). In terms of platform construction options, the data obtained show that direct antibody coating (PD#1 and PD#2) has significantly higher cell capturing capacity than the second antibody (PD#3) coating approach (See Table 1).

Petri dishes (PDs) were coated with 4B9 Antibody (Ab) old lot (PD#1), new lot (PD#2), or with 2nd-Ab (anti-mouse IgM) followed by incubation with unlabeled 4B9 antibody. Peripheral blood from 4 different first trimester pregnancies (about 30 mL) were washed, pooled, and used for fetal cell isolation in equal volumes. Isolated cells were stained for epsilon hemoglobin and counted manually using an inverted microscope.

### Experiment #2

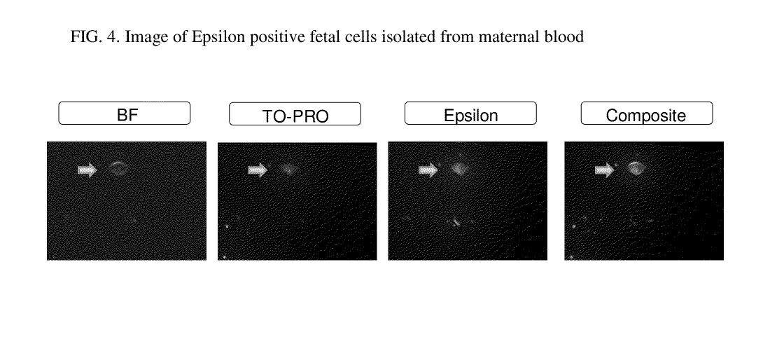

In the second experiment, five different glass microscope slides from three different manufacturers were first coated directly as well as indirectly with 4B9 and analyzed comparatively for their binding capacity with Cell-Free 4B9 ELISA. Glass slides demonstrating higher binding capacity were coated with biotinylated 4B9 via streptavidin coating (Slide #1), or unlabeled 4B9 via second antibody (Slide #2). Blood from another first trimester pregnancy (8 ml) was washed, resuspended to original volume, and incubated with slide #1 and slide #2 as above. The isolated cells were subsequently stained for fetal epsilon globulin and nuclei with TO-PRO. Microscope glass slides were coated with streptavidin or 2nd antibody followed by incubation with biotinylated 4B9 antibody (SA; Slide#1) or untouched 4B9 antibody (Slide#2). Peripheral blood from a single first trimester pregnancies (about 8 mL) was washed and used in equal volumes. Isolated cells were stained for epsilon hemoglobin and counter stained with TO-PRO. Isolated cells were counted manually using an inverted microscope. The numbers of epsilon-positive fetal cells isolated are summarized in Table 2 (see below) and representative cell images acquired microscopically are depicted in FIG. 4.

Consistent with previous findings, this blood sample also contained unprecedented high numbers of fetal cells that were readily isolated by the present invention. The number of epsilon-positive cells isolated by Slide #1 and Slide #2 were 98 (25/mL of maternal blood) and 203 (51/mL of maternal blood), respectively.

TABLE 2
Isolation of Fetal NRBC from a First Trimester Maternal Blood

| Solid | Capture | | Blood | Counter- | Hb detection | No. of double | No. of TO-PRO |
|---|---|---|---|---|---|---|---|
| Support | Ab | Blood | Vol/Test | stain | antibody | positive | positive only |
| Slide#1 | SA/Biotin- | 1^{st} | 4 mL | TO-PRO | AMCA | 98 | 7 |
| | 4B9(O) | Trimester | | | mAB human | | |
| | | | | | epsilon Hb | | |
| Slide#2 | 2nd-Ab- | l^{st} | 4 mL | TO-PRO | AMCA | 203 | 49 |
| | 4B9(O) | Trimester | | | mAB human | | |
| | | | | | epsilon Hb | | |

The observation that in these experiments, the number of isolated cells positive for TO-PRO but negative for epsilon was 7 by Slide #1 (non-specific binding of 7.1%) and 49 by Slide #2 (non-specific binding of 24%) suggests differential susceptibility of the various platforms to non-specific binding to nucleated cells that may be of maternal origin. Alternatively, some captured cells positive for TO-PRO only may be fetal NRBC cells that have lost epsilon globulin expression. Reportedly, definitive fetal erythroblasts that are potentially captured by 4B9 antibody are believed to be epsilon globulin negative (17, 22).

In comparison to results in Table 1, the lower number of fetal cells isolated by glass slides (Table 2) may be in part due to using smaller volume of maternal blood (4 mL vs. 10 mL), substantially smaller binding surface area of the glass vs. Petri-dish (by about 5 fold), and the fact that a different pregnancy sample was used. Simple extrapolation suggests that the number of fetal cells isolated by the two platforms would have been closer if similar sample volume and surface areas had been employed. However, the unprecedented fetal NRBC isolation sensitivity of 76-97% and isolation yield of 25-120 cells per mL of blood is significant achievement of a seemingly impossible task (17-18, 20-28). As such, the technology of the present invention fulfills the long-felt unmet need for a simple, reliable, and cost effective cell isolation technology for successful implementation of reliable non-invasive prenatal diagnostic tests.

### Cell Capture Platform and Application to Fish

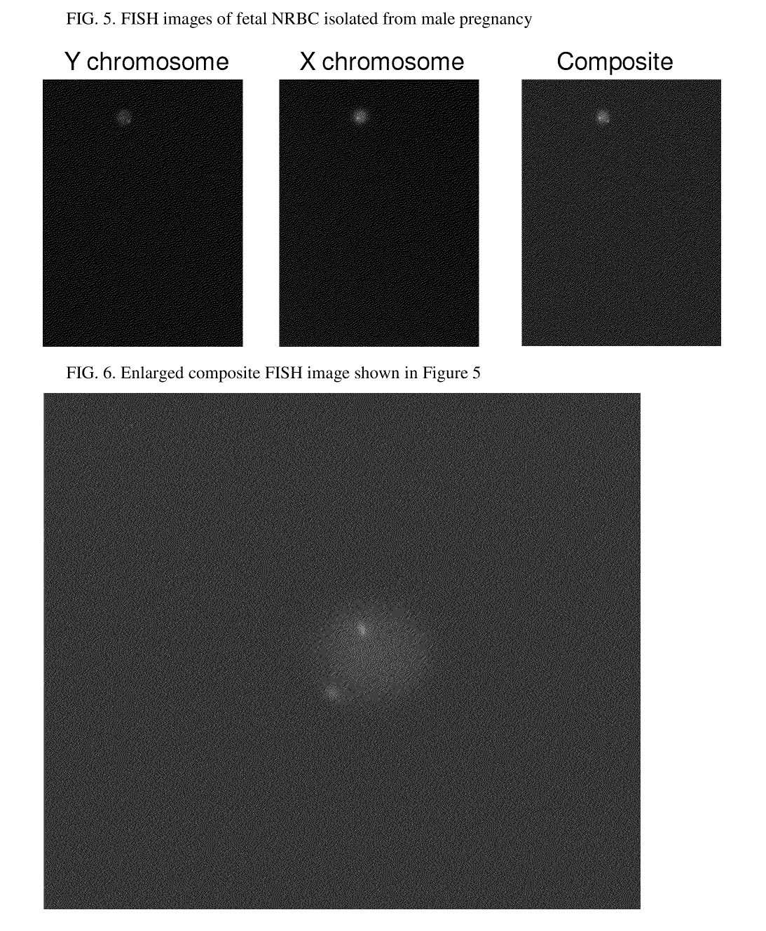

Blood (5 mL) from an ultrasound confirmed second trimester male pregnancy was washed and incubated with a glass slide directly coated with 4B9 antibody as described. Isolated cells were then processed accordingly and probed for detection of X and Y chromosome by FISH. As expected, the isolated fetal cells were specifically stained for both X (green) and Y (red) chromosomes. FIGS. 5 and 6 depict acquired images of the isolated cells, further confirming specificity and isolation efficiency of the present fetal cell isolation technology.

### Discussion

Despite the potential of circulating fetal NRBC cells as reliable predictors of fetal as well as maternal health and disease, progress in their isolation and analysis has been severely hampered by lack of efficient, simple, and reliable cell isolation methods. This persistent void has been consistently reflected in cumulating reports and, thus, scientific support that fetal NRBC in maternal blood are extremely rare and as such their successful isolation has been cited as formidable analytical and technical barrier (17, 18, 20-28). The novel discovery enabled by the use of the methods of the present invention that circulating numbers of fetal isolated from maternal blood are present in substantially higher numbers than ever expected is a strong testament of the unmet and differentiating efficiency of the present invention.

Based on theoretical considerations and new insight from a recent report (38), there may indeed be significantly higher numbers of fetal cells entering maternal circulation that have been previously thought. As the latter has been now demonstrated by the methods of the present invention, the long standing position on rarity of circulating fetal NRBC cells appears to be a direct reflection of inadequacies and inefficiencies of currently available technologies. The present invention has the potential to revolutionize the field of rare cell isolation in general and of fetal NRBC in particular by effectively fulfilling the current unmet needs for simple, reliable, and highly efficient rare cell isolation platform. The latter includes the consistent and highly efficiency isolation of rare cancer cells, an area that has been plagued by similar analytical and technical challenges (40).

In contrast to the inefficient multi-step approaches and strategies available to date, the present invention combines all of the required steps into a simple and seamless “one-step process” of fetal cell isolation. This novel approach is based on interfacing a convenient cell isolation platform such as glass slides, plastic containers, chambers, or wells with target cells of interest using a capture antibody against well defined cell surface biomarkers. Detection of specifically captured and isolated cells is then mediated by a detection antibody labelled with a readily detectable and/or quantifiable detection moiety.

The antibody-mediated sandwich-type cell isolation methods of the present invention have the novel and additional advantages of permitting the use of a single antibody for cell capture as well as detection, or combining a specific and/or non-specific capture antibody with one or more detection antibody that may be a reliable, though non-specific, identifier of target cells of interest. Because of its high cell isolation sensitivity and efficiency, the technology of the present invention is also applicable to development of quantitative methods for monitoring relative changes in the number of circulating cells that are known to occur in conditions such as Down syndrome, maternal complication of pregnancy such as preeclampsia, or a variety of human cancers (40). The quantitative cell isolation technology of the present invention, based on single-step isolation, detection, and counting the number of isolated cells per unit of the starting blood volume has additional advantages of simplicity and cost effectiveness.

The design of the technology of the present invention, focusing on solid-phase platforms that accommodate large surface area facilitate closer contact and thus enhanced capturing capacity and interaction of the solid-phase antibody with rare target cells of interest. This design, allowing the use of excess antibody planted on large binding surfaces has the advantage of promoting affinity independent interactions, enhanced reaction kinetics, and easy separation from unbound cells, while avoiding problems encountered by the commonly used microparticle-based cell separation strategies. Isolated rare cells can be then counted, analyzed in situ, and/or removed for downstream manipulation and analysis.

Each of the patents and references cited in this application are hereby incorporated herein by reference. In the event that there is an inconsistency between the teachings of one or more of the references incorporated herein and the present disclosure, the teachings of the present specification are intended. The examples provided in this specification are for illustration only and are not intended to limit the invention the full scope of which will be immediately clear to those of skill in the art.

### REFERENCES

1) Pierce B. Genetics: A conceptual approach (W.H. Freeman and company, 2008), third edition.
2) Driscoll D A, Gross S. Clinical practice. Prenatal screening for aneuploidy. N Engl J. Med. 360:2556-62, 2009.
3) Mujezinovic F, Alfirevic Z. Procedure related complications of amniocentesis and chorionic villus sampling: A systematic review Obstet Gynecol 110:687-94, 2007.
4) Tabor A, Philip J, Madsen M, Bang J, Obel E B, Norgaard-Pedersen B. Randomized control trial of genetic amniocentesis in 4606 low risk women. Lancet 1:1287-93, 1986.
5) Buscaglia M, Ghisoni L, Levi-Setti P E. Alpha-fetoprotein elevation in maternal serum after percutaneous umbilical blood sampling (PUBS). Prenat Diagn 16:375-76, 1996.
6) Thompson and Thompson Genetics in Medicine, sixth edition, chapter 9.
7) Thompson and Thompson Genetics in Medicine, sixth edition, chapter 5.
8) Nicolaides K H, Spencer K, Avgidou K, Faiola S, Falcon O. Multicentral study of first trimester screening for trisomy 21 in 75,821 pregnancies. Results and estimation of the potential impact of individual risk-oriented two-stage first trimester screening. Ultrasound Obstet Gynecol 25:221-26, 2005.
9) Evans M I, Hallahan T W, Krantz D, Galen R S. Met-analysis of first trimester Down syndrome screening studies: free beta hCG significantly outperforms intact hCG in a multi-marker protocol. Am J Obstet Gynecol 196:198-05, 2007.
10) Morris J K, Alberman E. Trends in Down syndrome live births and antenatal diagnosis in England and Wales from 1989 to 2008: analysis of data from the National Down Syndrome Cytogenetic Register. British Medical Journal 339, b3794.
11) ACOG Practice bulletin Clinical Management Guidelines for Obstetrician-Gynecologists, Number 7, January 2007).
12) Hahn S, Zhong X Y, Holzgreve W. Recent progress in non-invasive prenatal diagnosis. Semin Fetal Neonatal Med 13:57-62, 2008.
13) Hahan S, Lapaire O, Tercanli S, Kolla V, Hosli I. Determination of fetal chromosome aberrations from fetal DNA in maternal blood: has the challenge finally been met? Expert Reviews in Mol Med 13:e16, may 2011.
14) Schmorl C G, Pathologisch-Anatomische Untersuchungen uber Puerperal Eklampsie [pathological-anatomical explorations on puerperal eclapsia]. Leipzig, Germany:Verlag FCW Vogel.
15) Lo Y M, Corbetta N, Chamberlain P F, Rai V, Sargent I L, Redman C W, Wainscoat J S. Presence of fetal DNA in maternal plasma and serum. Lancet 350:485-87, 1997
16) Poon I L, Leung T N, Lau T K, Lo Y M. Presence of fetal RNA in maternal plasma. Clin Chem 46:1832-34, 2000.
17) Huang Z, Fong C-Y, Gauthaman K, Sukuma P, Choolani M, Bongso A. Novel approaches to manipulating fetal cells in the maternal circulation for non-invasive prenatal diagnosis of the unborn child. J C Biochem 112:1475-85, 2011.
18) Kavanagh D M, Keresaudy-Kerhoas M, Dhariwal R S, Desmulliez M P Y. Current and emerging techniques of fetal cell separation from maternal blood. J Chromat B 878:1905-11, 2010.
19) Bianchi D W. Feta cells in maternal blood: Feasibility for prenatal diagnosis. Br J Haematol 105:574-83, 1999.
20) Choolain M, O'Donoghue K, Talbert D, Kumar S, Robets I, Letsky E, R. Bennett P, M. Fisk N. Characterization of first trimester fetal erythroblasts for non-invasive prenatal diagnosis. Mol Hum Repro 9:227-35, 2003.
21) Calabrese G, Baldi M, Fantasia D, Sessa T, Kalantar M, Holzhauer C, Alunni-Fabbroni M, Palka G, Sitar G. Detection of chromosomal aneuploidies in fetal cells from maternal blood using single-chromosome dual-probe FISH analysis. Clin Genet Sept 2011.
22) Choolain M, O'Donnell H, Campagnoli C, Kumar S, Robets I, R. Bennett P, M. Fisk N. Simultaneous fetal cell identification and diagnosis by epsilon-globin chain immunophenotyping and chromosomal fluorescence in situ hybridization. Blood 98:554-7, 2001.
23) Ho S S Y, O'Donoghue K, Choolain M. Fetal cells in maternal blood: State of the art for non-invasive prenatal diagnosis. Ann Accad Med Singapore 32:597-604, 2003.
24) Kilpatrick M W, Tafas T, Evans M I, Jackson L G Antsaklis A, Brambati B, Tsipouras P. Automated detection of rare fetal cells in maternal blood: Eliminating the false-positive XY signals in XX pregnancies. A J Obstet Gynecol 190:1571-81, 2004.
25) Seppo A, Frisova V, Ichertovkin I, Kim Y, Evans M I, Antsaklis A, Nicolaides K H, Tafas T, Tispouras P, Kilpatrick M W. Detection of circulating fetal cells utilizing automated microscopy: potential for non-invasive prenatal diagnosis of chromosomal aneuploidies. Prenat Diagn 28:815-21, 2008.
26) Talasaz A H, Powell A A, Huber D E, Berbee J G, Roh K H, Yu W, Xiao W, Davis M M, Pease R F, Mindrinos M N, Jeffrey S S, Davis R W. Isolating highly enriched populations of circulating epithelial cells and other rare cells from blood using magnetic sweeper device. PANS 106:3970-75, 2009.
27) Kumo T, Tomizawa Y, Kita M, Takabayashi H, Tamiya E, Takamura Y. Concentration and extraction chip of fetal nucleated red blood cell (NRBC) by micro gap with diaphragm for fetal DNA diagnosis from maternal blood. 14th International Conference on Miniaturized Systems for Chemistry and Life Sciences. 3-7 Oct. 2010, Groningen, The Netherlands.
28) Cheng N, Liu F, Zhang L, Xu X-H, Gorthala S, Yana B. Enrichment of nuclear red blood cells by membrane KCC transporter with urea intervention. J Clin Lab Anal 25:1-7, 2011
29) John E Butler 1991, Immunochemistry of solid-phase immunoassay CRC Press)).
30) Pongsritasana T, Wongratanacheewin S, Prasetcharoensuk V, Sermswan R W. Isolation of fetal nucleated red blood cells from maternal blood using immunomagnetic beads for prenatal diagnosis. Asian pacific J Aller Immunol 24:65-71, 2006.
31) Khosravi M J, Diamandi A, Mistry J, Lee P D K. A non-competitive ELISA for human serum insulin-like growth factor-I. Clin Chem 42:1147-1154, 1996.
32) Khosravi M J, Diamandi A, Mistry J. Ultrasensitive immunoassay for prostate-specific antigen based on conventional colorimetric detection. Clin. Biochem. 28:407-414, 1995.
33) Khosravi M J, Diamandi A, Mistry J, Scorilas A. Insulin-like growth factor I (IGF-I) and IGF binding protein-3 in benign prostatic hyperplasia and prostate cancer. J. Clin Endocrinol Metab. 86:694-699, 2001.
34) Khosravi M J, Morton R C. Novel application of streptavidin-hapten derivatives as protein tracer conjugate in competitive-type immunoassays involving biotinylated detection probe. Clin Chem 37:58-63, 1991.
35). Khosravi J, Diamandis A, Mistry J, Krishna R G. The high molecular weight insulin-like growth factor binding protein complex: Epitope mapping, immunoassay, and preliminary clinical evaluation. J Clin Endocrinol Metab 84:2826-33, 1999.
36) Khosravi J, Krishna R G, Bodani U, Diamandi A, Khaja N, Kalra B, Kumar A. Immunoassay of serine-phosphorylated isoform of insulin-like growth factor (IGF) binding protein (IGFBP)-1. Clin Biochem 40:86-93, 2007.
37) American College of Medical Genetics. Technical and clinical assessment of fluorescence in situ hybridization: an ACMG/ASHG position statement. I. Technical considerations. Genetics in Medicine 2:356-361, 2000.
38) Sunami R, Komuro M, Tagaya H, Hirata S. Migration of microchimeric fetal cells into maternal circulation before placenta formation. Chimerism1: 66-68, 2010.
39) McGrath K, Kingsley P D, Koniski A D, Porter R L, Bushnell T P, Palis J. Enucleation of primitive erythroid cells generates a transient population of pyrenocytes in the mammalial fetus. Blood 111:2409-17, 2008.
40) Diamandis E P, Pantel K, Scher H I, Terstappen L, Lianidou E. Circulating cancer cells and their clinical applications. Clin Chem 57:1478-84, 2011.

We claim:

1. A method of isolating or enriching a rare cell from a biological fluid of a mammal, the method comprising:
(i) providing an antibody immobilized on a substrate, wherein the antibody binds a cell-surface antigen of the rare cell;
(ii) contacting the immobilized antibody with a sample of biological fluid, wherein the bodily fluid comprises the rare cell and a plurality of other cells;
(iii) incubating the immobilized antibody with the sample of bodily fluid under conditions suitable for binding of the antibody to the cell-surface antigen of the rare cell so as to form an antibody-rare cell complex; and
(iv) washing the antibody-rare cell complex to remove the unbound cells and provide an immobilized antibody-rare cell complex.

2. The method of claim 1, wherein the substrate comprises a glass or plastic surface.

3. The method of claim 2, wherein the glass or plastic surface is a surface of a plate, a petri-dish, a well, a microwell, a slide, a strip or a rod.

4. The method of claim 1, wherein the substrate is a particle or a bead.

5. The method of claim 4, wherein the particle or bead comprises a metal.

6. The method of claim 5, wherein the particle or bead is magnetic.

7. The method of claim 1, wherein the antibody is selective for a fetal cell surface antigen.

8. The method of claim 1, wherein the antibody is specific for a fetal cell surface antigen.

9. The method of claim 8, wherein the fetal cell surface antigen is a fetal nucleated RBC antigen.

10. The method of claim 9, wherein the antibody is antibody 4B9.

11. The method of claim 1, wherein the mammal is a human.

12. The method of claim 1, wherein the biological fluid is blood, plasma, amniotic fluid, urine, or a suspension of cells from a chorionic villus sampling (CVS) biopsy.

13. A method of detecting a rare cell in a biological fluid, the method comprising:
(i) providing a first antibody immobilized on a substrate, wherein the first antibody binds a first cell-surface antigen of the rare cell;
(ii) contacting the immobilized first antibody with a sample of biological fluid, wherein the bodily fluid comprises the rare cell and a plurality of other cells;
(iii) incubating the immobilized first antibody with the sample of bodily fluid under conditions suitable for binding of the first antibody to the first cell-surface antigen of the rare cell so as to form a first antibody-rare cell complex;
(iv) washing the first antibody-rare cell complex to remove the unbound cells and provide an isolated first antibody-rare cell complex;
(v) incubating the first antibody-rare cell complex with a second antibody that binds a second cell-surface antigen of the rare cell under conditions suitable for binding of the second antibody to the a second cell-surface antigen in order to form a first antibody-rare cell-second antibody complex and
(vi) detecting the second antibody in the first antibody-rare cell-second antibody complex and thereby detecting the presence of the rare cell in the sample of the bodily fluid.

14. The method of claim 13, wherein the biological fluid is blood, plasma, amniotic fluid, urine, or a suspension of cells from a chorionic villus sampling (CVS) biopsy.

15. The method of claim 13, the method further comprising a step of washing the first antibody-rare cell-second antibody complex so as to remove unbound second antibody between steps (v) and (vi).

16. The method of claim 13, wherein the first cell-surface antigen and the second cell-surface antigen are different.

17. The method of claim 13, wherein the cell-surface antigen and the second cell-surface antigen are the same.

18. The method of claim 13, wherein the first antibody is selective for a fetal cell surface antigen.

19. The method of claim 13, wherein the first antibody is for specific a fetal cell surface antigen.

20. The method of claim 19, wherein the first antibody is antibody 4B9.

21. The method of claim 13, wherein the second antibody is selective for a fetal cell surface antigen.

22. The method of claim 13, wherein the second antibody is specific for a fetal cell surface antigen.

23. The method of claim 22, wherein the second antibody is antibody 4B9.

24. The method of claim 22, wherein the second antibody is specific for fetal ε-globulin, CD36, CD71, or CD47.

25. The method of claim 13, wherein the second antibody is specific for glycophorin A or i-antigen.

26. The method of claim 13, wherein the first antibody is specific for CD36, CD71, or CD47.

27. The method of claim 13, wherein the second antibody is antibody 4B9.

28. The method according to claim 13, wherein the second antibody is labeled with a detectable label.

29. The method according to claim 28, wherein the detectable label is a fluorescent label, an enzyme label, a radioisotopic label, or a chemically reactive linking agent.

30. The method according to claim 13, wherein the second antibody is detected by a incubating the first antibody-rare cell-second antibody complex with a detectably labeled third antibody that specifically binds the second antibody under conditions suitable for antibody binding so as to form a first antibody-rare cell-second antibody-third antibody complex; washing the antibody-rare cell-second antibody-third antibody complex; detecting the detectably labeled third antibody; and thereby detecting the rare cell in the sample.

31. The method according to claim 30, wherein the detectably labeled third antibody is labeled with a fluorescent label, an enzyme label, a radioisotopic label or a chemically reactive linking agent.

32. The method according to claim 31, wherein the enzyme label is horse radish peroxidase or alkaline phosphatase.

33. A method of detecting a rare cell in a biological fluid, the method comprising:
(i) providing a first antibody immobilized on a substrate, wherein the first antibody binds a first cell-surface antigen of the rare cell;
(ii) contacting the immobilized first antibody with a sample of biological fluid, wherein the bodily fluid comprises the rare cell and a plurality of other cells;
(iii) incubating the immobilized first antibody with the sample of bodily fluid under conditions suitable for binding of the antibody to the cell-surface antigen of the rare cell so as to form a first antibody-rare cell complex and a plurality of unbound cells;
(iv) washing the first antibody-rare cell complex to remove the unbound cells;
(v) lysing the rare cells of the first antibody-rare cell complex to form a lysate that comprises a rare cell-specific nucleic acid sequence and incubating the lysed cells with a nucleic acid probe that is complementary to the rare cell-specific nucleic acid sequence under conditions suitable for hybridization of the nucleic acid probe with the rare cell-specific nucleic acid sequence in order to form a double stranded complex; and
(vi) detecting the double stranded complex and thereby detecting the presence of the rare cell in the sample of the bodily fluid.

34. The method of claim 33, wherein the biological fluid is a bodily fluid of a human or of an animal.

35. The method of claim 34, wherein the biological fluid is a bodily fluid of a human.

36. The method of claim 35, wherein the biological fluid is blood, plasma, amniotic fluid, urine, or a suspension of cells from a chorionic villus sampling (CVS) biopsy.

37. The method of claim 33, wherein the rare cell is a fetal cell.

38. The method of claim 33, wherein the double stranded complex is detected by fluorescence in-situ hybridization (FISH).

39. The method of claim 33, wherein the rare cell-specific nucleic acid sequence is characteristic of a chromosomal abnormality.

40. The method of claim 39, wherein the chromosomal abnormality is a single gene abnormality.

41. The method of claim 40, wherein the chromosomal abnormality is characterized by a single nucleotide polymorphism (SNP).

42. The method of claim 33, wherein the rare cell-specific nucleic acid sequence is characteristic of a predisposition to a carcinoma.

43. A kit for capture, detection or isolation of a rare cell from a biological fluid, the kit comprising:
(i) a first antibody immobilized on a substrate wherein the antibody is specific for a cell-surface antigen of the rare cell; and
(ii) a buffer solution suitable for antigen antibody binding.

44. The kit according to claim 43, suitable for antibody binding to a rare cell in the biological fluid, wherein the biological fluid is blood, plasma, amniotic fluid, urine, or a suspension of cells from a chorionic villus sampling (CVS) biopsy.

45. The kit according to claim 43, wherein the substrate comprises as a glass or plastic surface.

46. The kit according to claim 43, wherein the cell-surface antigen of the rare cell is a cell-surface antigen of a fetal cell.

47. The kit according to claim 46, wherein the fetal cell is a fetal nucleated red blood cell (NRBC).

48. The kit according to claim 47, wherein the first antibody is 4B9.

49. The kit according to claim 43, wherein the cell-surface antigen of the rare cell is a cell-surface antigen of a cancer cell.

50. The kit according to claim 49, wherein the first antibody is specific for a cell surface antigen specific to the cancer cell.

51. The kit according to claim 43, further comprising a nucleic acid specific fluorescent dye.

52. The kit according to claim 43, further comprising a second antibody specific for second cell surface antigen of the rare cell, wherein the second antibody is not immobilized.

53. The kit according to claim 52, wherein the rare cell is a fetal cell.

54. The kit according to claim 53, wherein the fetal cell is a fetal nucleated red blood cell (NRBC).

55. The kit according to claim 54, wherein the second antibody is 4B9.

56. The kit according to claim 54, wherein the second antibody is an antibody specific for CD36 or CD71.

57. The kit according to claim 54, wherein the second antibody is a mixture of antibodies specific for CD36 and CD71.

58. The kit according to claim 54, wherein the second antibody is an antibody specific for glycophorin-A or i-antigen.

59. The kit according to claim 52, wherein the rare cell is a cancer cell.

60. The kit according to claim 52, wherein the second antibody is detectably labeled.

61. The kit according to claim 60, wherein the detectably labeled second antibody is labeled with a fluorescent label, an enzyme label, a radioisotopic label or a chemically reactive linking agent.

62. The kit according to claim 52, wherein the second antibody is specific for CD36, CD71, CD47, glycophorin-A, i-antigen, or fetal epsilon globulin.

63. The kit according claim 43, wherein the substrate is suitable for use for direct hybridization analysis.

64. The kit according claim 43, further comprising a nucleic acid probe complementary to a gene of the rare cell.

65. The kit according claim 64, wherein the nucleic acid probe is suitable for fluorescence in situ hybridization (FISH) analysis of the rare cell.

66. A method of estimating the number of rare cells per unit of a biological fluid of a mammal, wherein the method comprises:
(i) providing an antibody immobilized on a substrate, wherein the antibody binds a cell-surface antigen of the rare cell;
(ii) contacting the immobilized antibody with a known unit sample of biological fluid, wherein the bodily fluid contains a plurality of rare cells and a plurality of other cells;
(iii) incubating the immobilized antibody with the unit sample of bodily fluid under conditions suitable for binding of the antibody to the cell-surface antigen of the rare cell so as to form antibody-rare cell complexes;
(iv) washing the antibody-rare cell complexes to remove the unbound cells and provide immobilized antibody-rare cell complexes; and
(v) detecting the number of immobilized antibody-rare cell complexes in the sample and thereby estimating the number of rare cells per unit of the sample fluid.

67. The method of claim 66, wherein the biological fluid is blood, plasma, amniotic fluid, urine, or a suspension of cells from a chorionic villus sampling (CVS) biopsy.

68. The method of claim 66, wherein the number of rare cells per unit of the sample fluid outside of a normal range is diagnostic or prognostic for a disease or condition, or is indicative of the clinical status of a disease or condition.

69. The method of claim 68, wherein the disease or condition is a fetal genetic disease or condition.

70. The method of claim 68, wherein the disease or condition is a maternal complication of pregnancy.

71. The method of claim 70, wherein the maternal complication of pregnancy is preeclampsia.

72. The method of claim 68, wherein the disease or condition is cancer.

73. The method of claim 66, wherein the rare cell is a fetal cell.

74. The method of claim 73, wherein the fetal cell is a fetal nucleated red blood cell (NRBC).

75. The method of claim 66, wherein the immobilized antibody-rare cell complexes are detected with a cell nucleus-specific stain.
